(19) United States
(12) Patent Application Publication
(10) Pub. No.: US 2025/0049301 A1
(43) Pub. Date: Feb. 13, 2025

(54) SHAPED ENDOSCOPY SUPPORT DEVICE INSERT

(71) Applicant: ColoWrap, LLC., Durham, NC (US)

(72) Inventors: James HATHORN, Carrboro, NC (US); Wayne HENDERSON, Carrboro, NC (US)

(21) Appl. No.: 18/799,990

(22) Filed: Aug. 9, 2024

Related U.S. Application Data
(60) Provisional application No. 63/519,156, filed on Aug. 11, 2023.

(51) Int. Cl. A61B 1/00; A61B 1/01

(52) U.S. Cl. CPC A61B 1/0014; A61B 1/01

(57) ABSTRACT

A method and apparatus for helping to direct endoscope movement and/or applying pressure to a patient's abdomen during an endoscopy procedure. An apparatus for placement at an abdomen of a patient in connection with an endoscopy procedure, includes a main body having a curved surface, wherein the main body is configured to extend across and curve along at least a portion of the abdomen of the patient. The apparatus also includes at least one contour in an exterior of the main body, wherein the at least one contour is shaped to guide movement of an endoscope during one or more of insertion or withdrawal of the endoscopy procedure.

### CROSS REFERENCE TO RELATED APPLICATION(S)

This application claims the benefit of and priority to U.S. Provisional Application Ser. No. 63/519,156, entitled “Endoscopy Support Device Insert” and filed on Aug. 11, 2023, which is expressly incorporated by reference herein in its entirety.

### INTRODUCTION

A colonoscopy is an example of an endoscopy procedure including an examination of the large intestine or colon through the use of a colonoscope. A colonoscope is a flexible, tube-like inspection device having a camera at its end. Colonoscopies are performed for a variety of medical reasons including detection of inflamed tissue, ulcers, abnormal growths or polyps, and colorectal cancer. Colonoscopy is increasingly used as a screening tool to detect colorectal cancer.

During a colonoscopy, as an example of an endoscopy procedure, a colonoscope is inserted into a patient's rectum and then advanced to the beginning of the colon (an area known as the cecum) in order to examine the lining of the large intestine. The efficiency and accuracy of this procedure is largely dependent on the ease with which the colonoscope can be advanced. During the procedure, the colon may become over-distended or flopped in unnatural directions creating loops that hinder the advancement of the colonoscope and resulting in patient discomfort, longer examination times, and potentially inaccurate or incomplete screenings.

Currently, the difficulty in advancing the scope is addressed by the application of manual pressure by a technician to manually support the patient's colon. The application of manual pressure is time-consuming and varies depending on the particular technician's strength, technique, endurance, and training. In order to apply differential pressure or to change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure places a physical toll on the technician.

### SUMMARY

In an aspect of the disclosure, a method and apparatus for applying pressure to the abdomen of a patient is provided to case the passage of an endoscope during procedures used to examine the bowels including colonoscopy, sigmoidoscopy, and enteroscopy. Aspects presented herein exert both broad, uniform lower abdominal pressure as well as additional, location-specific pressure upon specific parts of the colon including the sigmoid and transverse, and/or small bowel to assist with the advancement, withdrawal, and/or visualization as part of an endoscopy, colonoscopy, sigmoidoscopy, or enteroscopy procedure. Aspects presented herein may assist in preventing and reducing intestinal looping, eliminating the need for the application of manual pressure, improving patient safety, comfort, and satisfaction, and preventing musculoskeletal injury to endoscopy healthcare providers. Aspects may improve visualization and/or withdrawal portions of the procedure.

Aspects include an insert that can be used in connection with an support device that applied pressure through compression. The insert may help in the application of the compression from the device to the abdomen of the patient and may assist in applying a more consistent amount of support to patients of varying body mass indexes. In some aspects, the insert may assist in maintaining a position of the sigmoid and/or transverse regions of the colon during an endoscopy procedure. In some aspects, the insert may be shaped to assist in guiding an endoscope lumen during an insertion procedure.

In some aspects, an apparatus for placement at an abdomen of a patient in connection with an endoscopy procedure, comprising: a main body having a curved surface, wherein the main body is configured to extend across and curve along at least a portion of the abdomen of the patient; and at least one contour in an exterior of the main body, wherein the at least one contour is shaped to guide movement of an endoscope during one or more of insertion or withdrawal of the endoscopy procedure.

In some aspects, a method of applying an insert at a patient in connection with an endoscopy procedure, includes: placing a shaped insert between the patient and at least a portion of an elastic compression wrap, wherein the shaped insert includes: a main body having a curved surface configured to extend across and curve along at least a portion of an abdomen of the patient; and at least one contour in an exterior of the main body; applying at least the portion of the elastic compression wrap over the shaped insert; and maintaining the shaped insert at the abdomen of the patient during at least one of endoscope insertion, imaging, endoscope withdrawal, or a post endoscopy time period.

Additional advantages and novel features of aspects of the present invention will be set forth in part in the description that follows, and in part will become more apparent to those skilled in the art upon examination of the following or upon learning by practice thereof.

### BRIEF DESCRIPTION OF THE DRAWINGS

FIG. 1A is a schematic view of a colon with an endoscope (also known as a colonoscope for colonoscopy procedures) partially inserted therein.

FIG. 1B is a schematic view of a colon in which a sigmoid loop has developed due to an attempt to advance the endoscope against an unsupported colon wall;

FIG. 1C is a schematic view of a colon showing the application of manual pressure to the colon to facilitate insertion of an endoscope.

FIG. 2 is an illustration of an unfastened endoscopy band device with an elastic secondary strap capable of retaining tension and exerting directed force to the colon, including the sigmoid, traverse, and cecal regions, stretched and fastened to the primary wrap.

FIG. 3A is a perspective view of the endoscopy band device depicted in FIG. 2 as it appears when applied to a patient, in accordance with aspects of the present invention.

FIGS. 3B and 3C illustrate examples of a technician applying manual pressure to the abdomen of a patient.

FIG. 4 illustrates a method of use of an endoscopy band device with an elastic secondary strap capable of retaining tension and exerting directed force for an endoscopy procedure.

FIG. 5 is an illustration of a portion of an endoscopy band having multiple secondary straps.

FIG. 6 is an endoscopy band with multiple secondary straps applied to a patient.

FIG. 7 illustrates a pelvic region of a patient, including an iliac crest.

FIG. 8A and FIG. 8B illustrate cross-section views of an insert applied between a patient and a compression wrap.

FIG. 9 illustrates a side view of an example insert.

FIG. 10 illustrates a top view of an example insert.

FIG. 11 illustrates an end view of an example insert, e.g., from a tapered end.

FIG. 12 illustrates a side view of an example insert.

FIG. 13 illustrates an example of a pressure sensor placed between a patient and an example endoscopy support device, e.g., a compression wrap.

FIG. 14 illustrates an example of an insert for use with an endoscopy support device.

FIG. 15 illustrates an example of an insert placed between the primary wrap and the secondary straps of the example endoscopy support device.

FIGS. 16 and 17 illustrate examples of an insert placed between the primary wrap and under the secondary straps of the example endoscopy support device.

FIGS. 18-24 illustrate an example of an endoscope moving through a colon with support and guidance provided by the insert.

FIG. 25 illustrates an example sequence of an endoscope moving through a colon with support and guidance provided by the insert.

FIG. 26A and FIG. 26B illustrates example aspects of methods of use of an insert with an endoscopy support device during an endoscopy procedure.

FIGS. 27A-C illustrate perspective views of an example insert.

### DETAILED DESCRIPTION

Aspects include an insert that can be used in connection with a support device that applied pressure through compression. The insert may help in the application of the compression from the device to the abdomen of the patient and may assist in applying a more consistent amount of support to patients of varying body mass indexes. In some aspects, the insert may assist in maintaining a position of the sigmoid and/or transverse regions of the colon during an endoscopy procedure. In some aspects, the insert may be shaped to assist in guiding an endoscope lumen during an insertion procedure.

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

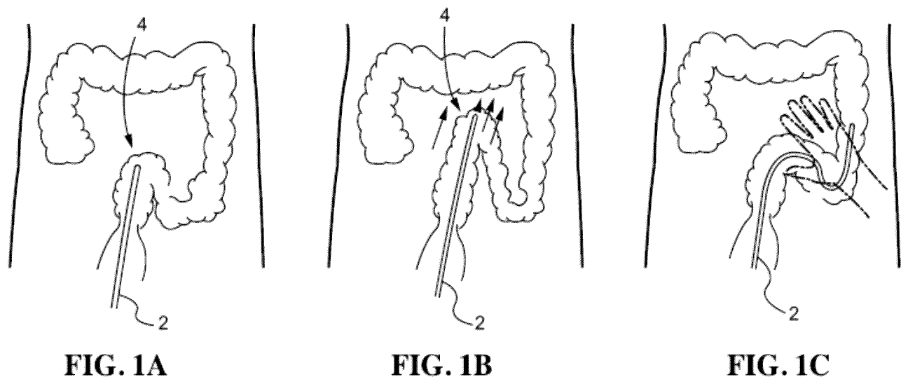

FIGS. 1A-1C, illustrate a sequence of steps of a colonoscopy, as one example of an endoscopy procedure. In FIG. 1A, a colonoscope 2 is inserted into the patient's rectum and advanced forward through the length of the colon. As the operator passes the colonoscope through the sigmoid region of the colon 4, the colonoscope may become impinged and cause distention and looping of the anatomy, as shown in FIG. 1B. The distention causes discomfort to the patient and increases the time required for the colonoscopy. In order to reduce the distended or looped area, a technician may apply manual pressure to abdomen of the patient. Among other examples, the technician may be a nurse, assistant, or other staff member. For example, the pressure may be applied by a nurse or surgical assistant as shown in FIG. 1C.

The application of manual pressure is time-consuming and places a physical toll on the technician. The effectiveness of the manual pressure varies depending on the particular technician's strength, technique, endurance, and training. In order to apply differential pressure and change the orientation of the colon within the body, the technician may roll the patient from the left side to a supine or to a prone position, which can be a difficult task with a sedated patient. The application of manual pressure and movement of the patient in order to support the patient's colon and advance the colonoscope during the procedure may lead to injury of the patient or of the technician.

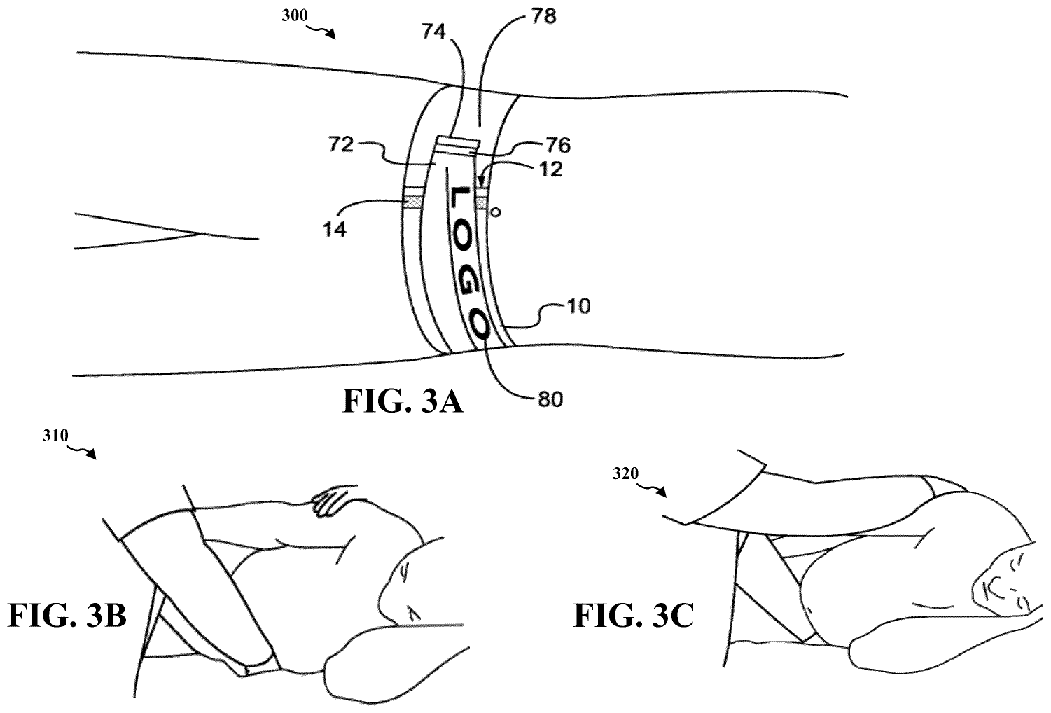

Many patients undergo colonoscopy while placed in the left lateral decubitus position on the stretcher or operating table. FIG. 3B and FIG. 3C illustrate examples 310 and 320 of a technician applying manual abdominal pressure when the patient is placed in a left lateral decubitus position. Additional information about the use of such manual pressure can be found in Prechel J A, Hucke R. Safe and effective abdominal pressure during colonoscopy: forearm versus open hand technique. Gastroenterol Nurs 2009; 32:27-30; quiz 31-2, the entire contents of which are incorporated herein by reference. In applying manual pressure, the technician may reach over the patient from the opposite side of the table and to deploy pressure by placing their hands against the patient's sigmoid colon and then leaning backwards, using their bodyweight for leverage to exert force. While these methods are generally effective at generating pressure, they have also been identified as a causative factor for the high rate of work-related injuries among endoscopy nurses and staff. Physicians performing colonoscopy suffer work-related musculoskeletal injury at a particularly high-rate as well. The most frequent site of physician injury is the right upper extremity which experiences peak torque forces when while operators are attempting to advance the scope through (a looping) sigmoid colon. Additional details can be found in Spanarkel M, Hathorn J P. Looping During Colonoscopy: A Major, Implied Cause of Injury Among Endoscopy Healthcare Providers and a Proposed Solution, 2013, the entire contents of which are incorporated herein by reference.

Aspects described herein may similarly be applied for other endoscopic procedures such as, but not limited to, sigmoidoscopy and retrograde enteroscopy procedures. Sigmoidoscopy is an examination of only the lower part of the colon, from the anus to the descending colon. An endoscope is inserted into the lower part of the colon. Enteroscopy is an examination of the small bowel. During retrograde enteroscopy, an endoscope is inserted in the anus and passed through the colon and the cecum and into the small bowel. Successfully navigating the loop-prone sigmoid region is necessary to complete both sigmoidoscopy and retrograde enteroscopy and thus aspects described herein can be used to help facilitate colonoscopy, sigmoidoscopy, retrograde enteroscopy, and other endoscopic procedures. The small bowel is part of the intestine between the stomach and the colon. The small bowel comprises three segments, the duodenum, the jejunum, and the ileum. It may be difficult to advance an endoscope in order to visualize the patient's entire small bowel due to the length, tortuosity/redundancy of the small bowel.

The teachings of each of U.S. application Ser. No. 17/180,676, entitled Endoscopy Band with Visual Indicator to Assist Placement, and filed on Feb. 19, 2021; U.S. application Ser. No. 16/818,877, entitled Endoscopy Band with Sigmoid Support Apparatus, and filed on Mar. 13, 2020; U.S. application Ser. No. 15/256,019, entitled “METHOD AND APPARATUS FOR ENHANCED VISUALIZATION DURING ENDOSCOPY,” and filed on Sep. 2, 2016; U.S. Provisional Application No. 62/214,747, entitled “IMPROVED BOWEL STABILITY AND ENHANCED VISUALIZATION DURING ENDOSCOPY” and filed on Sep. 4, 2015; and U.S. application Ser. No. 14/575,860, entitled “ENDOSCOPY BAND WITH SIGMOID SUPPORT APPARATUS,” and filed on Dec. 18, 2014; U.S. application Ser. No. 13/344,715, entitled “METHOD AND APPARATUS FOR TENSILE COLONOSCOPY COMPRESSION,” and filed on Jan. 6, 2012; U.S. Provisional Application Ser. No. 61/917,469, entitled “COLONOSCOPY BAND WITH SIGMOID SPLINT” and filed on Dec. 18, 2013; U.S. Provisional Application Ser. No. 61/944,658 entitled “ENDOSCOPY BAND WITH SIGMOID SUPPORT APPARATUS” and filed on Feb. 26, 2014; U.S. Provisional Application Ser. No. 62/978,797, entitled “Endoscopy Band With Visual Indicator to Assist Placement” and filed on Feb. 19, 2020, are expressly incorporated by reference herein in their entirety.

In order to prevent looping and assist in insertion and/or withdrawal of an endoscope, and/or to improve imaging during an endoscopy procedure, a compression device may be placed on the patient. The device may include a primary elongated band or wrap of sufficient length for placement around a patient's lower abdomen. The primary wrap may include a closing mechanism, such as a hook and loop fastener material to secure the device around the patient and to apply an amount of broad support and compression. The device may also include one or more secondary straps that enable the technician to adjust the amount of compression applied by the device.

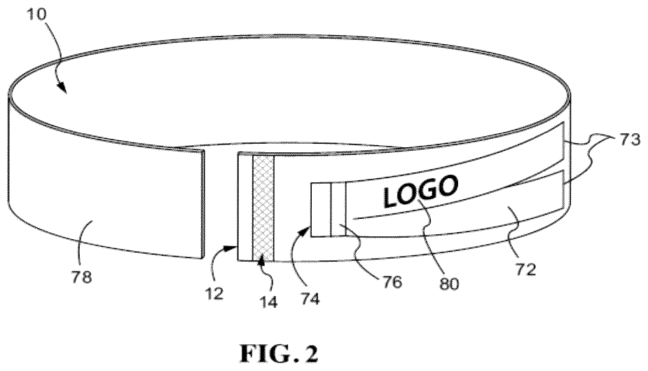

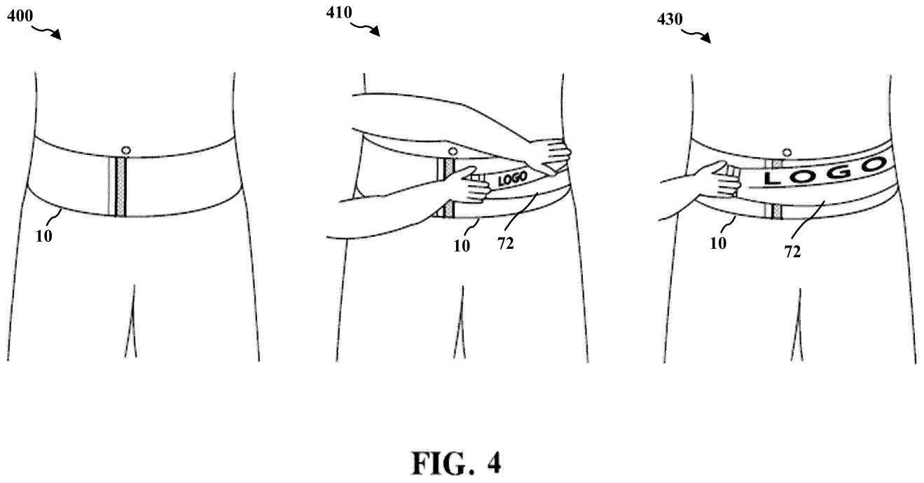

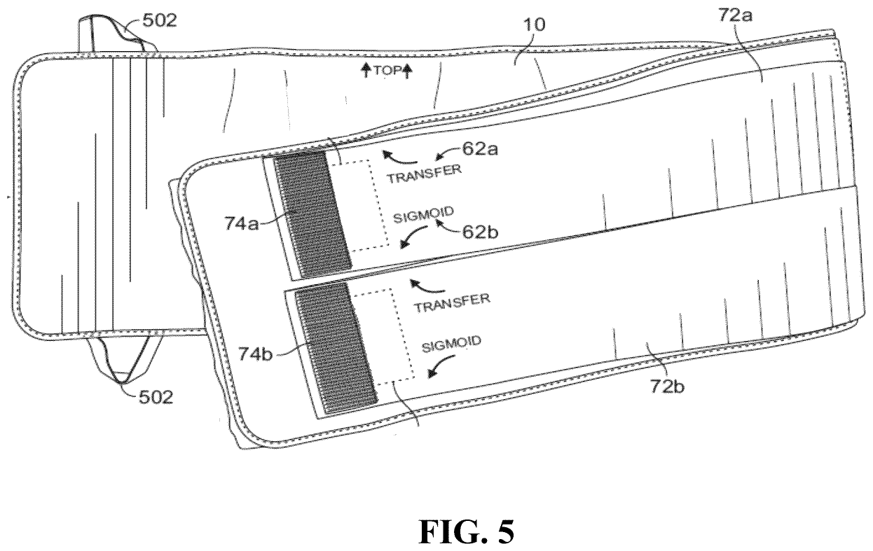

FIG. 2 illustrates an example device including a primary elongated band or wrap 10 of sufficient length for placement around a patient's lower abdomen. A closing mechanism 12 may be provided at the end of the primary band to secure the device around the patient so that it provides the desired amount of broad support and compression. A handle 14 may be sewn onto the exterior of one or both ends of the primary wrap to assist in fastening and closure. FIG. 4 illustrates an example application 400 showing a primary elongated band or wrap 10 wrapped around an abdomen of a patient. FIG. 4 shows at 410 and 420, a secondary strap 72 being pulled, or stretched, to apply added compression to at least a portion of the abdomen. FIG. 5 illustrates a portion of an example wrap 10 having handles 502 provided at an end of the wrap 10. The handles may be used by the technician to stretch the wrap and apply a desired degree of compression when fastening the primary wrap 10 around the patient. In some aspects, an extension component, or offset piece, may extend from the primary wrap 10, and the handles 502 may be formed as a loop of material that is placed at the respective extensions. In some aspects, an angled placement of the handles or finger loops may improve the leverage of the user in pulling the edge of the primary wrap underneath the opposite edge in order to apply compression before fastening the opposite edge to the primary wrap, e.g., using a hook and loop or hook and pile type fastener, in order to maintain the compression on the abdominal region of the patient.

The primary wrap 10 may have a circumferential length between approximately 15 and 75 inches in order to accommodate varying abdominal girths in patients. The width of the primary wrap 10 may be between 6 and 10 inches, although variations having widths of between 3 and 20 inches may be used depending on the size of the patient and to accommodate special circumstances such as an abdominal hernia or a large pannus. For example, the primary wrap may be configured to have a width that allows it to be fastened around the patient's lower abdomen with the upper edge of the wrap just below the umbilicus, or near the umbilicus, and the bottom edge of the wrap along or close to the pubic line. The width of the primary wrap may be selected so as not to be so large that the upper edge conceals the umbilicus and additional areas of the abdomen above the umbilicus. This configuration may be made in order to avoid pressure on the diaphragm or stomach, as pressure on the diaphragm and stomach during colonoscopy can increase the risk of oxygen deprivation and aspiration events, respectively, due to the fact that the patient is generally sedated during the procedure. The primary wrap 10 may comprise, entirely or in part, a flexible, bio-compatible foam, rubber, neoprene, polyester, nylon, non-woven or woven fabric, mesh fabric, synthetic fabric, microfiber fabric, silicon or vinyl plastic, or any other materials generally known to be used in medical fabrics and goods. The primary wrap 10 may be composed of both elastic and inelastic materials. In one example, the primary wrap 10 may comprise multiple layers laminated together. For example, the primary wrap 10 may comprise a neoprene layer and an outer fabric layer laminated on the neoprene layer. The fabric layer may enable the secondary strap to be removably fastened along the length of the primary wrap and may also enable visual indicators to be printed on the fabric, such as a nylon loop fabric. The primary wrap may also comprise an inner layer laminated on the neoprene. The inner layer may comprise a fabric or other material that is selected for skin sensitivity. For example, the inner layer may comprise a fabric or other material that is latex free, biocompatible, and/or skin sensitive. The inner layer may be the layer that is applied against the skin of the patient. In some aspects, the primary wrap may comprise 3D spacer fabrics, which may provide compression, elasticity, cushioning, breathability, air permeability, thermal insulation, temperature regulation, flexibility with resistance to bending/rolling, good draping, adjustable vapor transport, good aging qualities, surface and wash resistance, sterilization capabilities, and diverse surface design capabilities. A 3D spacer fabric may comprise a manufactured textile structure in which two fabric layers are connected by a layer of pile threads, which may be referred to as pile yarns.

The primary wrap may comprise two or more sections that vary in material type. In one example, a first set of one or more sections may be composed of a flexible, elastic or semi-elastic, medium-thickness, latex-free neoprene with thin polyester or nylon glued to its interior and exterior sides. Among others, this material may be capable of providing broad, firm, yet comfortable support to the patient's abdominal region. Another set of one or more sections of the primary wrap 10 may comprise a relatively inelastic material, such as a woven fabric. The inelastic section may be provided at the location into which or under which an insert is placed. Upon deployment of the device, the inelastic section may be positioned in the patient's lower left abdominal quadrant, over the sigmoid region.

For the primary wrap to provide appropriate general compression and support, it may be important that the wrap remain flat against the body when fastened around the abdomen. This is notable because certain materials and designs have a tendency to roll-up when stretched or wrapped around the abdomen, particularly when the device is being applied to patients with a large pannus. To prevent roll-up from occurring, aspects of the invention may include reinforcements to ensure that the primary abdominal wrap remains flat against the body when used in patients of varying body sizes. This may be accomplished by the application of serge stitching along the edges of the primary abdominal band.

The primary wrap may also accommodate an insert or attachment that provides specific support to one or more areas of the colon including the sigmoid, transverse, and cecal regions as well as the splenic and hepatic flexures. When the device is securely fastened, the secondary strap, the insert, or both, may be pushed, pulled, or otherwise pressed into the body in a manner that serves to support or ‘splint’ one or more areas of the colon including the sigmoid, transverse, and cecal regions as well as the splenic and hepatic flexures. Aspects described herein may be designed to provide broad lower abdominal support, and additional direct force to one or more areas of the colon including the sigmoid, transverse, and cecal regions as well as the splenic and hepatic flexures of a patient undergoing colonoscopy.

The primary wrap 10 may be placed around the patient's lower abdomen and secured using a closing mechanism 12 consisting of a strip of VELCRO® or hook material 13 placed on the interior of the wrap 10 close to the location of the handle 14 on the opposite side. This hook strip 13 may be fastened to the exterior side of the opposite end of the primary wrap 10.

The addition of one or more appendages on the exterior of the primary wrap facilitates the application of additional directed force, e.g., without requiring adjustment of the primary wrap. The appendages may comprise one, or multiple, straps attached on one side to the edge or edges of non-elastic section. FIGS. 2, 3A, and 4 illustrate an example with a single secondary strap.

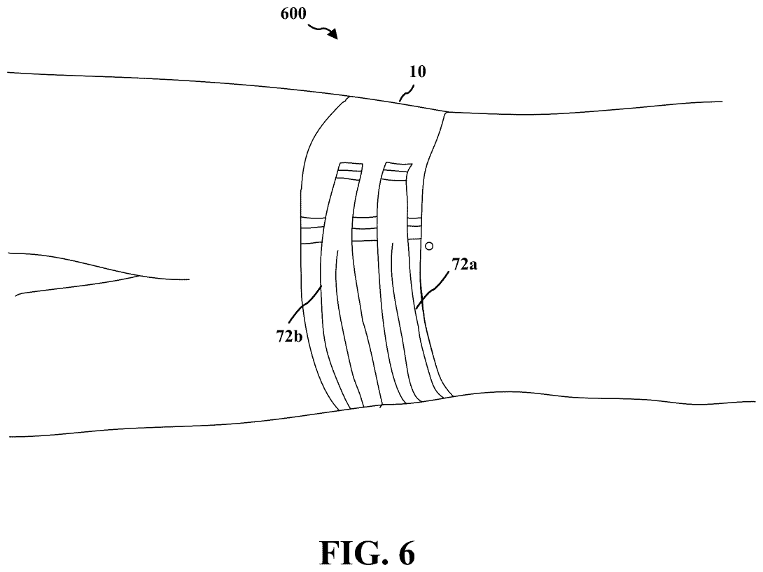

FIG. 5 illustrates an example having multiple secondary straps 72a, 72b. FIG. 6 illustrates an example 600 of a device having multiple secondary straps that is placed on a patient. On the unattached end of these straps, there may be a handle, e.g., 74a, 74b shown in FIG. The handle or loop near the end of the strap may provide greater case to a technician, or added security, in pulling the clastic strap to apply targeted, additional compression. The straps 72a, 72b may be pulled horizontally along the exterior of the primary wrap, and fastened using the closing mechanism securely enough to maintain tension. The one or more straps may comprise an elastic material that is configured to be stretched and fastened to the primary wrap in order to apply targeted compression through elastic contraction of the material. The amount of targeted compression may be adjusted through adjustment of the position at which the removable end is coupled to the primary wrap, such as through a hook and loop type fastener. VELCRO® is an example of a hook and loop type fastener. In some examples, the straps may be secured to an inelastic section of the primary wrap, and the tension generated by fastening these straps may cause additional compression of the inelastic section and/or an insert toward the body of the patient. In some aspects, the device may include visual labels 62a, 62b, e.g., including text and/or arrows that assist with placement and/or application of targeted compression.

As depicted at 300 in FIG. 3A, the secondary strap 72 may allow nurses and technicians to easily adjust and readjust the force on a particular region of the patient's abdomen, such as the sigmoid colon and/or the transverse colon among other example regions, from the location in the procedure or operating room that these staff members typically occupy, relative to how patients are often positioned during an endoscopy procedure. The endoscopy compression device described herein additionally eliminates the need for the nurse or assistant to provide manual abdominal compression, thereby reducing their risk of musculoskeletal injury. Additional, manual compression may be applied along with compression from the device. The device is designed to be quickly and easily removed should the need arise.

The secondary strap 72 may be coupled, e.g., sewn or permanently fastened, to a portion of the wrap that is configured for placement over a left side of the patient's lower abdomen. The portion of the secondary strap 72 that is coupled to the primary wrap may be referred to as the secured end and may be non-removably coupled to the strap, such as sewn or formed as an extension of the primary wrap material. The strap can be configured to be pulled from left to right, e.g., to stretch across the lower abdomen and/or left lower abdominal quadrant from the left side of the patient's body towards right side of the patient's body. This placement of the secondary strap 72 may enable the compression to be easily adjusted as the patient lies on their left side during the procedure. As the strap pulls from left to right across the lower abdomen, additional leverage and compression may be generated by the patient's body when the strap is in place. As the strap stretches from left to right cross the patient's abdomen, the level of compression generated by the device can easily be adjusted during the procedure, e.g., while the patient is lying on their left side.

The secondary strap 72 may attach to the exterior side 78 of the primary wrap 10, with a closing mechanism 74 that allows the strap, e.g., to be fastened to the exterior side 78 of the primary wrap 10. In an aspect, the closing mechanism 74 of the secondary strap 72 may comprise a hook strip 75 on the inside of the secondary strap 72 that is capable of fastening anywhere along the exterior side 78 of the primary wrap 10. In this example, the exterior side 78 of the primary wrap 10 may comprise a hook-compatible material to which the hook strip 75 can be fastened and remain fastened while the secondary strap is stretched and under tension. The secondary strap 72 may be constructed of an elastic or semi-elastic material that is capable of retaining tension when stretched horizontally and fastened to the exterior side 78 of the primary wrap 10 using the closing mechanism 74. The secondary strap 72 may comprise the same or different materials than the materials comprised in the primary wrap 10. The secondary strap 72 may comprise one or more layers of materials. In an example, the secondary strap 72 may comprise an elastic strap the entire length of which equals approximately twice the intended length of the secondary strap 72. As illustrated, the secondary strap may taper along a length of the strap 72. In the example in FIG. 2, the clastic strap may be doubled-over, and both ends may be sewn to the exterior 78 of the primary wrap 10 along the same vertical line 73, creating the horizontal ‘V’ appearance of the secondary strap 72. Doubling-over the material composing the secondary strap can serve to increase the breadth and force generated by the secondary strap, while preventing the incurrence of additional materials costs that would be associated with using a taller, single-layered strap instead. The midpoint of the secondary strap may be positioned approximately over the patient's lower left quadrant once it is stretched and fastened. In some aspects, the secondary strap 72 may be a unitary piece having at least one tapering edge.

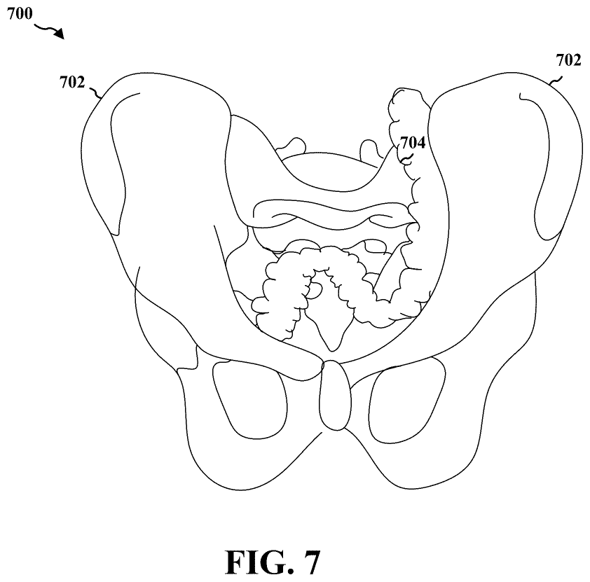

In some aspects, patient anatomy may limit the compression applied by the device. FIG. 7 illustrates an example 700 showing that the iliac crest 702 that can hold the device away from the colon 704 to which compression is intended to be applied. For example, in patients with a low BMI, the iliac crest may extend beyond the abdomen and may hold the primary wrap and secondary strap away from compressing the abdomen.

In order to apply pressure, e.g., compression, to the abdomen, one or more inserts may be placed between at least a portion of the device and the patient's abdomen. In some aspects, the insert may be placed between the patient's abdomen and the primary band, for example. The insert may be configured to communicate compression from the primary band and/or secondary strap to the abdomen of the patient. The insert can be used in combination with the compression device to improve the efficacy of the compression.

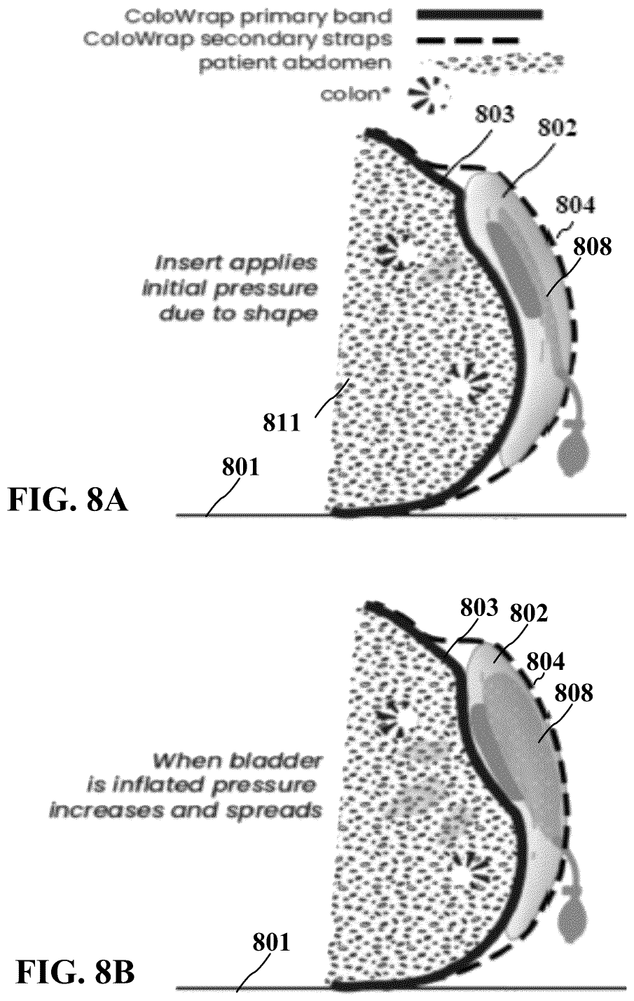

FIG. 8A and FIG. 8B illustrate cross-section views of an insert applied between a patient and a compression wrap. As shown, in some aspects, the insert 802 may be inserted between a primary wrap 803 and a secondary strap 804. FIG. 8A and FIG. 8B illustrate a portion of cross section of the patient's abdomen with the compression wrap and insert applied with the patient lying on their side on an exam table 801. Although the insert 802 is illustrated between the primary wrap 803 and the secondary strap 804, in other aspects, the insert 802 may be positioned between the patient's abdomen 811 and the primary wrap 803. FIG. 8A illustrates an example of the insert with a deflated, or uninflated bladder 808, and FIG. 8B illustrates an example of the insert 802 having an inflated bladder 808. The insert may be configured as a shaped, contoured insert, that is configured for a specific firm application of pressure so as to minimize or prevent looping of a colonoscope or similar device. When an iliac crest, e.g., 702, of a patient with a lower BMI prevents a compression device, e.g., with a band that surrounds the abdomen of the patient, from applying compression, the insert 802 can fill an area between the compression device and the abdomen of the patient and help to communicate the pressure through compression to the area of the colon. The contoured shaping is configured to help maintain the position of a sigmoid and/or transverse colon, e.g. as a scope in inserted, and/or to guide the scope in the intended direction. The counter-pressure applied by the combination of the compression device and the insert may allow the colonoscope to traverse along the colon.

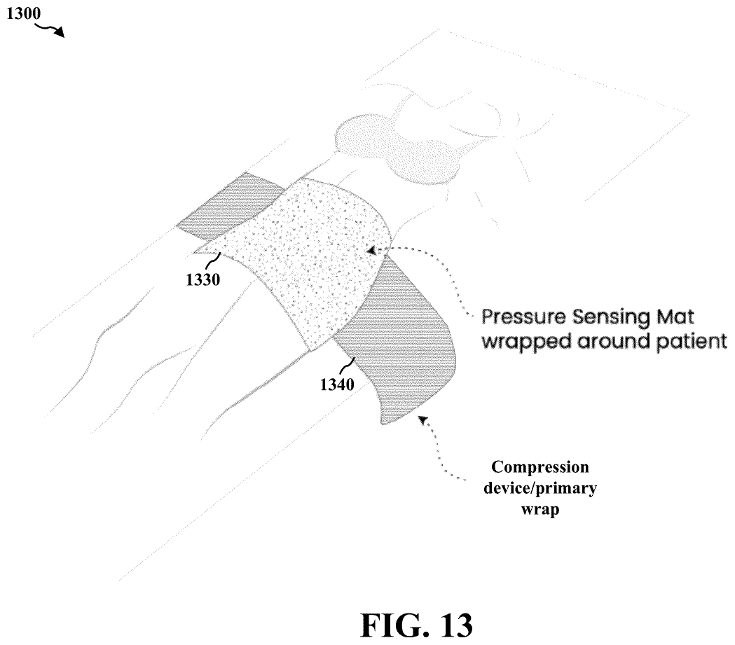

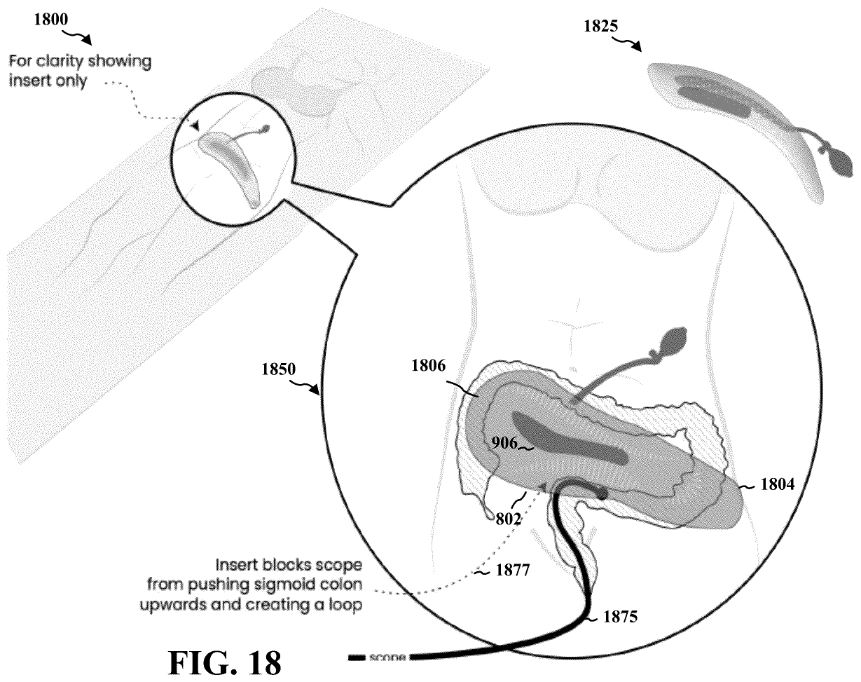

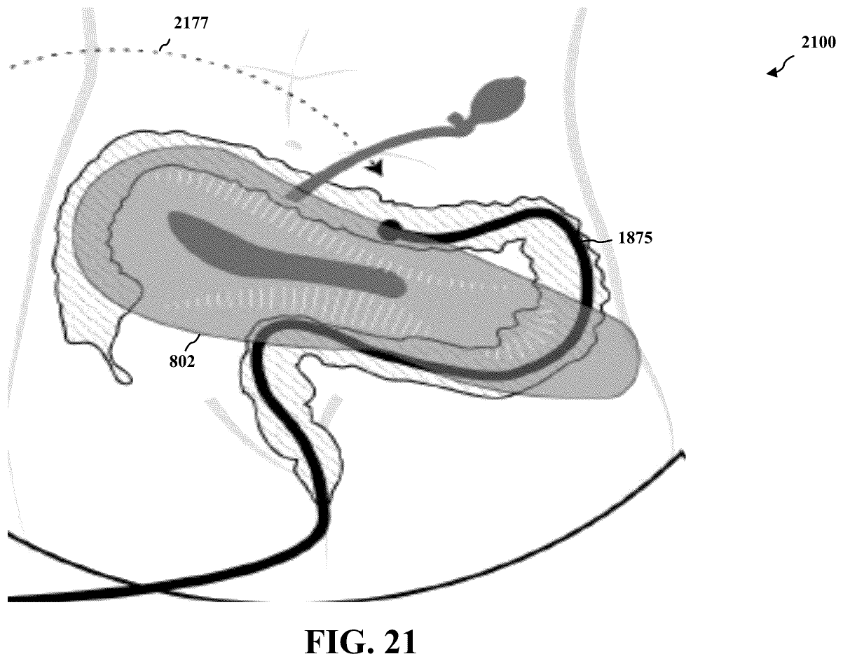

The contoured shape of the insert may correspond to portions of the colon (e.g., sigmoid, transverse). The contoured shape of the insert, when utilized to apply pressure to the abdomen of a patient, may be designed to catch, block, stop, and/or guide the endoscope or provide a support surface that counters pressure applied by the endoscope onto the colon to prevent the colon from looping while the endoscope is being advanced within the colon, as shown for example in FIGS. 13-25. For example, as shown in FIG. 18, the insert 802 may help the compression device to apply a pressure and block the endoscope from pushing the sigmoid colon upwards and creating a loop. In another example, as shown in FIG. 21, as the endoscope moves through the transverse colon, the insert may block the endoscope from pushing the colon downwards and forming an upper loop.

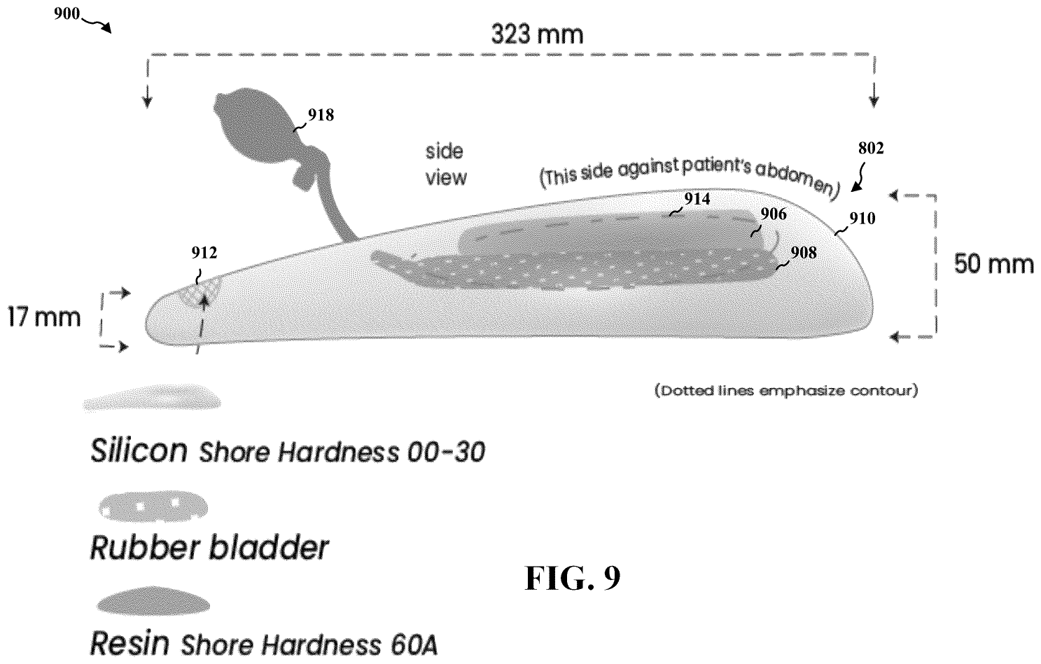

As shown in FIG. 8A and FIG. 8B, the insert may comprise multiple components. The insert may comprise a core component 906 (or inner core), an inflatable component 908, and an outer component 910 (. The core component 906 and the inflatable component 908 may be housed within the outer component 910 which may be referred to as an outer housing or outer material). FIG. 9 illustrates a side view 900 of the insert 802. Although the outer component 910 may be opaque, semi-transparent, or transparent, the inner components are shown in FIG. 9 in order to illustrate their placement. The material of the core component 906 may have a higher hardness than the outer component 910. In some aspects, the insert may be referred to as a dual hardness, or dual density device. In some aspects, the core component 906 may include a material of shore hardness 60A, and the outer component 910 may have a shore hardness of 00-30. The outer component 910 may include a biocompatible material, such as silicon. For example, the outer component 910 may include a material that can be placed directly against the skin on a patient's abdomen without adverse effects.

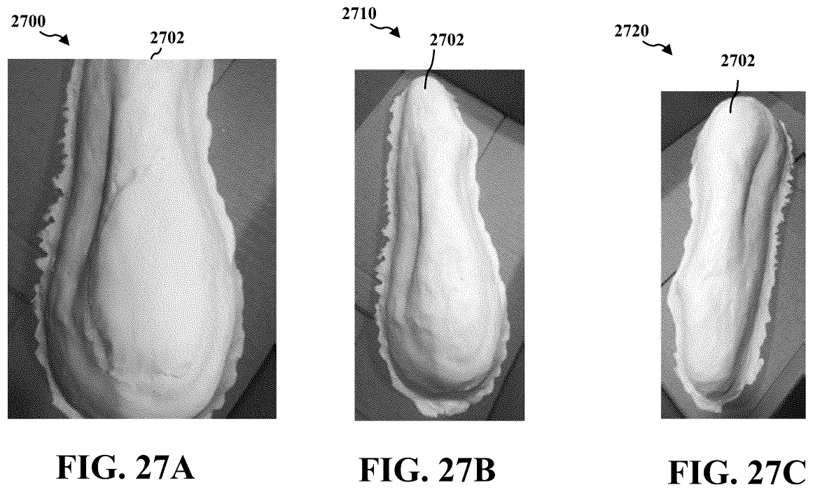

The core component 906 may be shaped or contoured to correspond with, align with, or follow a shape of the colon (e.g., sigmoid, transverse) of the patient. The dashed line 914 in FIG. 9 illustrates an area of contouring. FIGS. 27A-C illustrate views 2700, 2710, and 2720 of an example of mold 2702 for forming, or manufacturing, the insert 802, which show the contoured shape of the exterior of the insert.

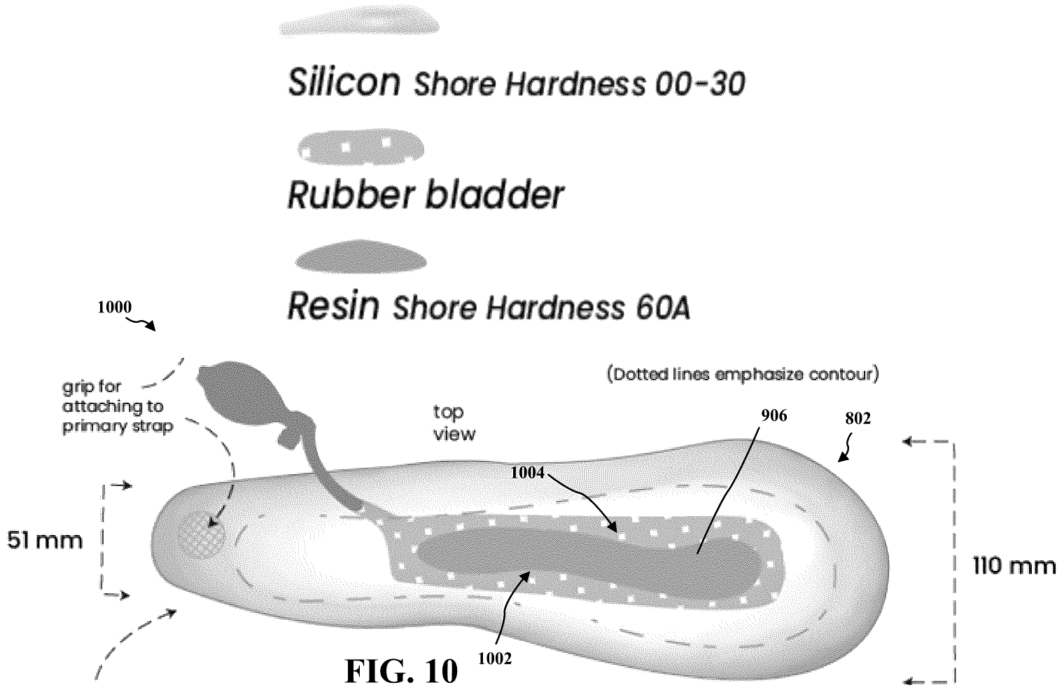

FIG. 10 illustrates a view 1000 of the insert 802 showing that the core component 906 may have a lower indentation or groove that is shaped to correspond with a portion of the sigmoid colon, such that the lower indentation or groove provides a surface and/or localized pressure to guide or assist the colonoscope to traverse along the sigmoid colon. In addition, the core component 906 may have an upper indentation or groove that is shaped to correspond with a portion of the transverse colon, such that the upper indentation or groove provides a surface and/or localized pressure to guide or assist the colonoscope to traverse along the transverse colon.

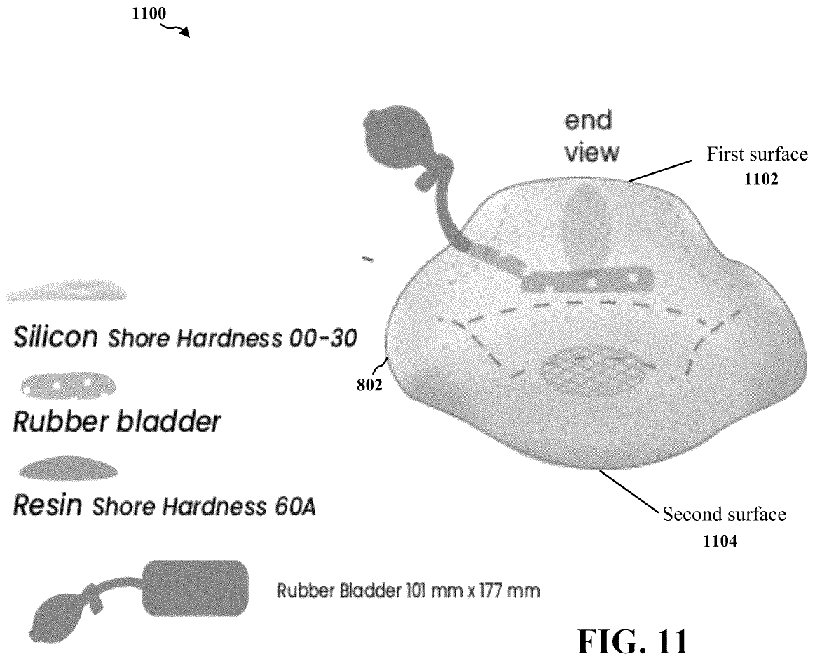

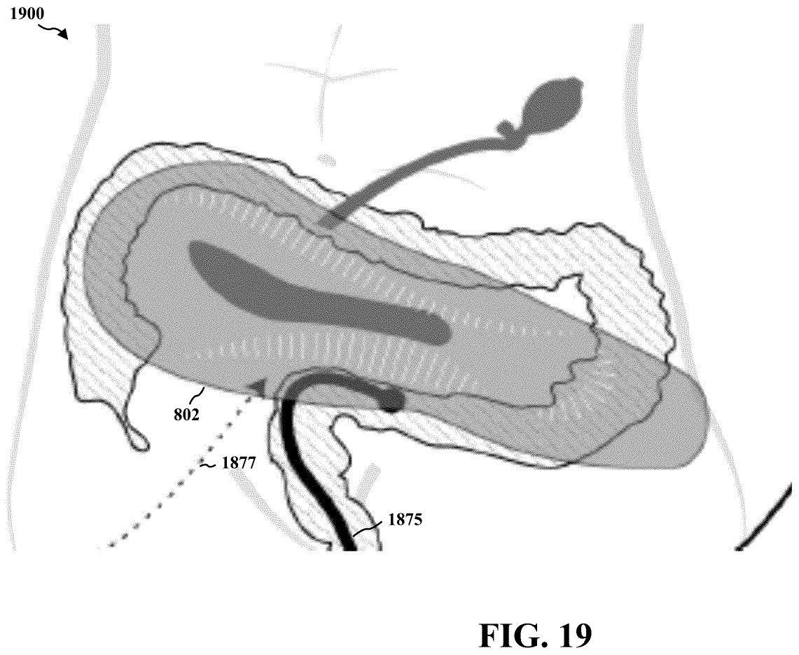

FIG. 10 shows a first region 1002 of the core component 906 may be shaped to stop a sigmoid colon area from moving out of position, or looping, when an endoscope 1875 is inserted, e.g., as shown in FIG. 18 and FIG. 19. The curved shaped of the first region 1002 may guide the endoscope 1875 in the desired direction, as shown with the arrow 1877. FIG. 10 also shows a curved shaping in a second region 1004, which may provide similar positioning and guidance of the endoscope when traversing the transverse colon, e.g., as shown in FIGS. 21-24. For example, FIG. 21 illustrates a view 2100 with an arrow 2177 showing the contoured shape of the insert 802 helping to guide the endoscope 1875 as it begins to be inserted through the transverse colon area. The contoured shaping of the core component 906 and/or the outer component 910 may help to cup the sigmoid colon or the transverse colon as an endoscope 1875 is moved through the colon. In addition to the shaping, the hardness of the materials of the outer component 910 and the core component 906 may be selected to redirect the endoscope rather than block the endoscope 1875. For example, a softer material may provide some resistance without providing a blocking action that may be provided by a harder material. The view in FIG. 10 shows a transparent view through the insert 802 from a side of the device that is applied toward the abdomen of the patient. For example, FIG. 11 illustrates a transparent end view of the insert. In some aspects, the outer material may be opaque. The first surface 1102 may be positioned toward an abdomen of the patient, and the second surface 1104 may be positioned away from the abdomen of the patient when the insert is placed to provide endoscopy support.

The inflatable component 908, e.g., bladder, may be configured to apply added pressure onto the abdomen of the patient. For example, the inflatable component 908 may comprise a bladder that may be inflated that applies pressure onto the abdomen of the patient such that the core component may provide localized pressure, as discussed herein, to assist or allow the colonoscope to traverse the colon during a colonoscopy procedure. The inflation may adjust the shape, size, and/or stiffness of the insert to adjust the guidance of the endoscope. The core component 906 may apply an added pressure inward towards the abdomen of the patient as the bladder, e.g., 906, is inflated. In some instances, the insert 802, when applied between the patient and the compression wrap, may apply an initial pressure due in part to the shape of the insert. However, when the bladder is inflated, pressure exerted on the patient may increase and spread which may assist the endoscope to traverse the colon. For example, the inflatable component 908 may include a bladder that is inflated or decreased via a hand pump 918. The bladder may comprise a rubber material, as an example. In some aspects, an insert 802 may be configured without an inflatable component 908. In some aspects, the insert may be configured to receive an insertable inflatable component 908 so that a user can adjust or modify the insert based on the individual anatomy of a particular patient.

The outer component may be shaped to assist in the localized application of pressure. For example, with reference to FIGS. 9-12, the outer component may have a first surface and a second surface. The first surface may be applied onto the patient and contact the compression wrap, as shown for example in FIGS. 8A, 8B, and 13-17. The first surface may comprise a grip surface, e.g., as shown at 912, that helps to maintain the positioning of the insert on the patient. For example, the grip surface may comprise a hook and loop material, pointed extensions or protrusions, an adhesive, or the like that may fasten or adhere to the compression wrap so as to ensure that the insert does not move or become displaced. In some aspects, the first surface may have the grip surface on at least part of the first surface.

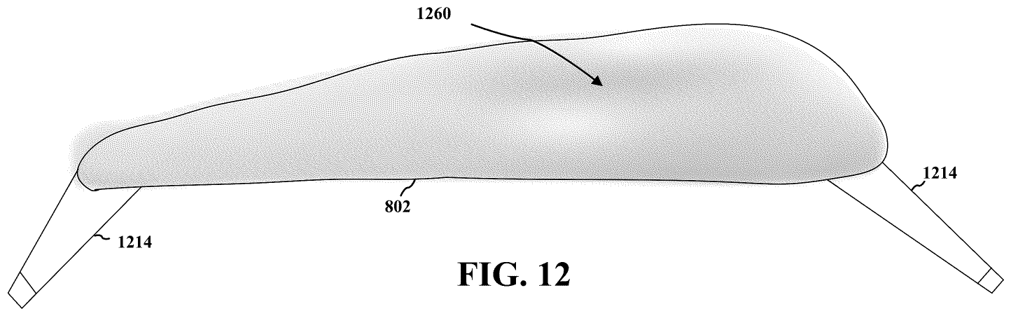

The first surface may comprise a raised or contoured surface, as shown for example in the end view 1100 in FIG. 11. Similar to FIGS. 8-10, FIG. 11 is a transparent view showing the internal components of the insert. The first surface of the insert is placed toward the abdomen of the patient such that the insert is substantially aligned with the colon of the patient. The raised or contoured surface of the first surface may be configured to prevent the colon from looping while the colonoscope is traversing the colon. The raised or contoured surface of the first surface may assist in the core component in providing the localized pressure and shape that is designed to catch the colonoscope or provide the support surface that counters pressure applied by the colonoscope onto the colon and prevent the colon from looping while the colonoscope is being advanced within the colon. FIG. 12 shows an opaque view of the insert and shows the contoured shaping 1260 of the outer surface of the insert.

The outer component, or outer housing, of the insert 802 may further be shaped to assist in the application of the localized pressure. For example, with reference to FIG. 10, the outer component may have a tapered shape such that one side of the outer component may be wider than the opposite side. With reference to FIG. 10, the outer component may have a width of 110 mm at one side and may taper to have a width of 51 mm at the opposing side. The tapering of the insert may correspond to the colon of the patient which may assist in providing the pressure in a desired region of the abdomen of the patient, which may assist in providing the desired pressure to assist the colonoscope traverse the colon. FIG. 18 illustrates an example placement of the insert 802 showing the larger end 1806 of the outer housing placed at a right hip of the patient, and a tapered end 1804 of the insert placed toward a left side of the patient's abdomen.

The disclosure is not intended to be limited to the specific dimensions described herein. The specific dimensions and materials are provided as an example to illustrate the concept. For example, the insert may have many different while employing the concepts disclosed herein. In some aspects, multiple device sizes may be provided, and a particular size may be selected for a particular patient.

In some aspects, the inflatable component 908 may be configured to be inserted within the insert, such that the pressure component is not permanently housed within the outer component of the insert. This may allow for differently sized pressure components to be utilized in connection with the insert, which may allow to accommodate for different body types.

In some aspects, the insert may comprise extensions or straps that may assist in maintaining the position or location of the insert on the abdomen of the patient, e.g., even in the absence of a compression device and/or prior to securing with a compression device. The extensions or straps may be configured to assist in compressing the insert onto the patient. For example, the extensions or straps may extend from opposing edges or ends of the outer component of the insert. The insert may be held into place due in part to the extensions or straps. FIG. 12 illustrates an example showing a single strap 1214 provided at each end of the insert. Although an example is shown of a single strap, multiple straps may also be provided at each end of the insert. In some aspects, an end of the strap that is not secured to the insert may include a hook material that fastens to an outer surface of a compression wrap (e.g., a primary wrap). In some aspects, the ends of the straps may include an adhesive so that the straps may be secured to the patient's abdomen. In instances where the pressure component is an inflatable bladder, the bladder may be inflated to a point where localized pressure on the patient is not localized or is being applied outwards on a mechanism (e.g., straps) that cover the insert and intended to hold the insert in place. The outward pressure applied onto the straps holding the insert in place may reduce the localized pressure being applied onto the abdomen. As such, the extensions or straps on the opposing edges of the outer component of the insert may pull into the body and fasten the insert to the compression wrap, thereby maintaining the application of the desired pressure on the abdomen of the patient.

In some aspects, the first surface of the outer component may comprise a channel. The channel may assist in providing the localized pressure. For example, when the bladder of the pressure component is inflated, the channel may be configured to flare out into the abdomen which may assist in providing the localized pressure in a desired region of the abdomen. The channel may assist in the orientation of the pressure when the bladder is inflated. In some aspects, the channel may be comprised on the second surface, while in some aspects, the channel may be comprised on the first surface and the second surface.

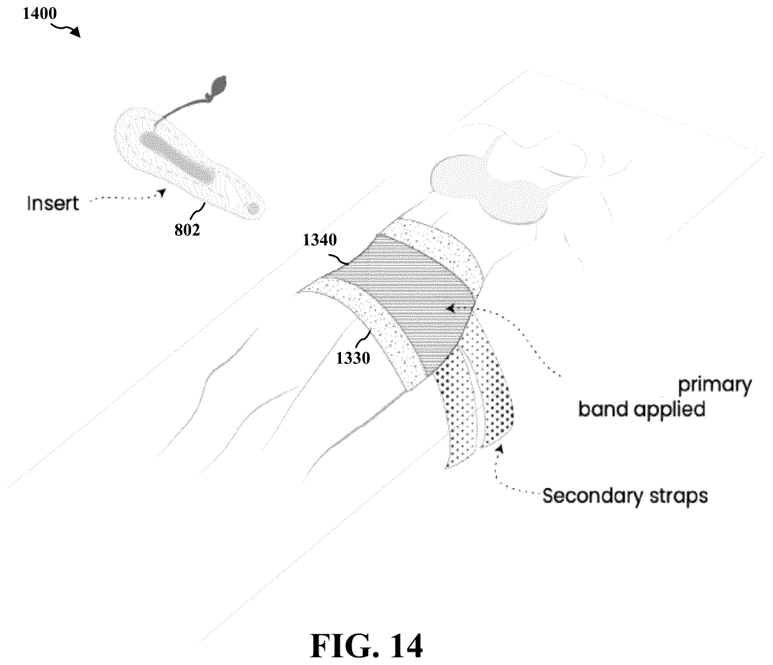

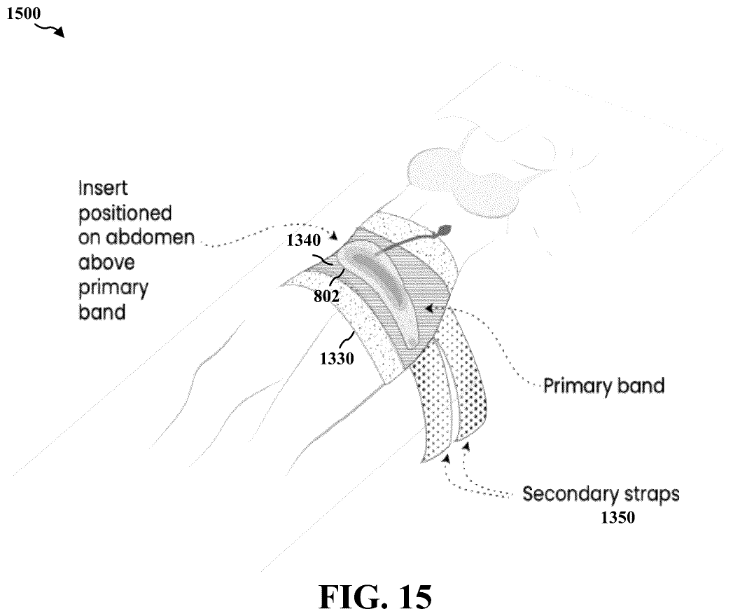

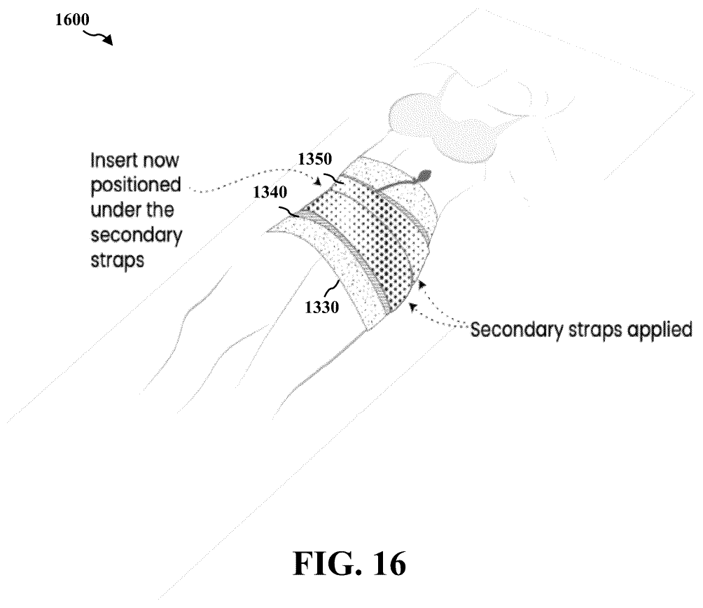

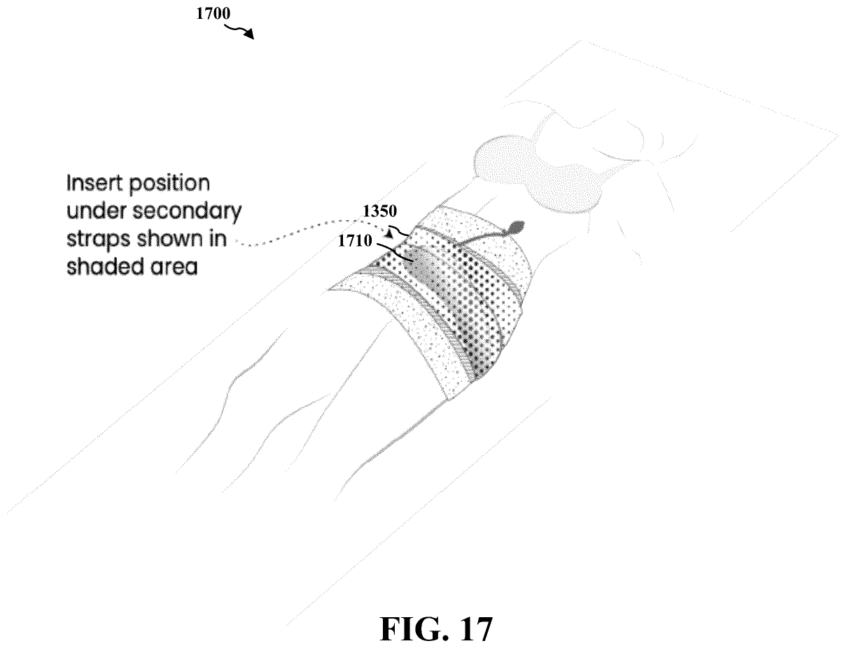

FIGS. 13-17 illustrate an example placement of the insert together with a compression device. FIG. 13 illustrates a view 1300 showing that a pressure sensitive mat 1330 may be wrapped around a patient prior to application of the insert 802 and compression device 1340. The compression device 1340 may include any of the aspects described in connection with FIGS. 2-6. FIG. 14 shows a view 1400 in which a primary band of the compression device 1340 is wrapped and secured around the abdomen of the patient. FIG. 15 illustrates an example 1500 in which the insert is positioned on the abdomen of the patient prior to application of the secondary straps 1350. FIG. 16 illustrates a view 1600 in which the secondary straps 1350 are secured over the insert 802 so that the insert is positioned between the primary wrap and the secondary straps 1350. In some aspects, the insert 802 may be applied to the abdomen of the patient prior to application of the primary wrap. FIG. 17 illustrates an example view 1700 showing a shaded location 1710 of the insert 802 positioned under the secondary straps 1350 of the compression device.

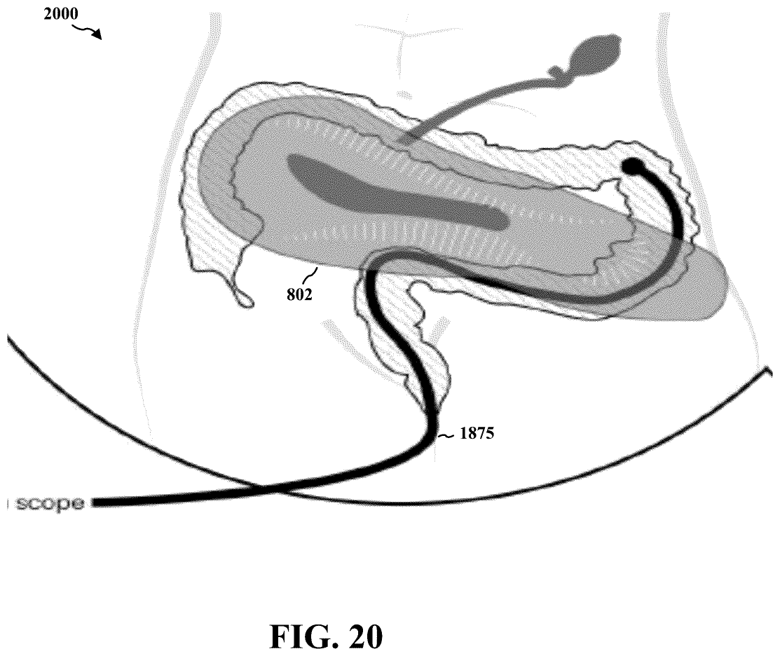

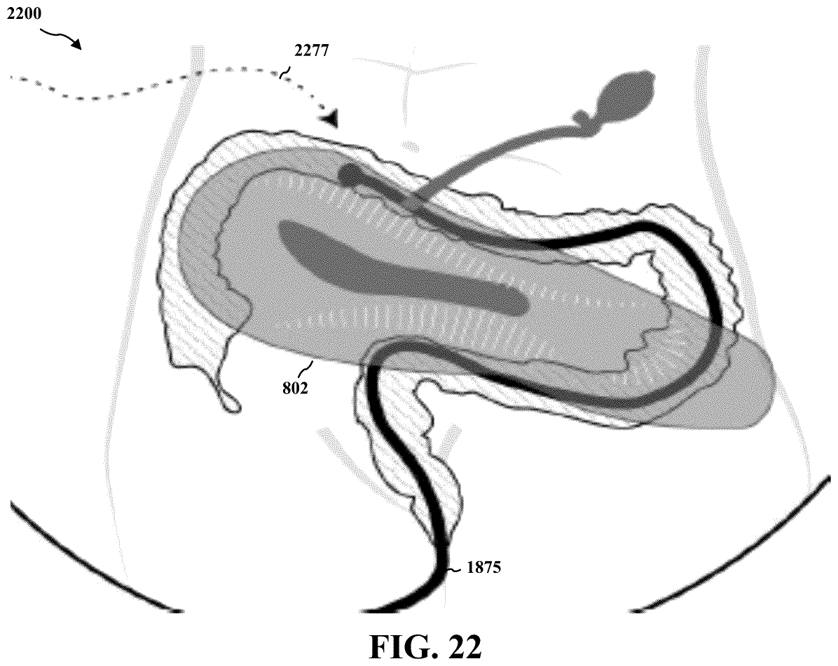

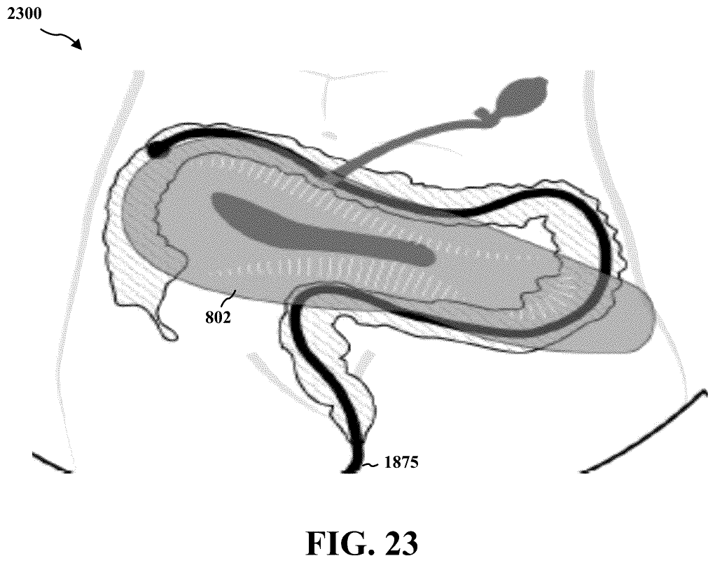

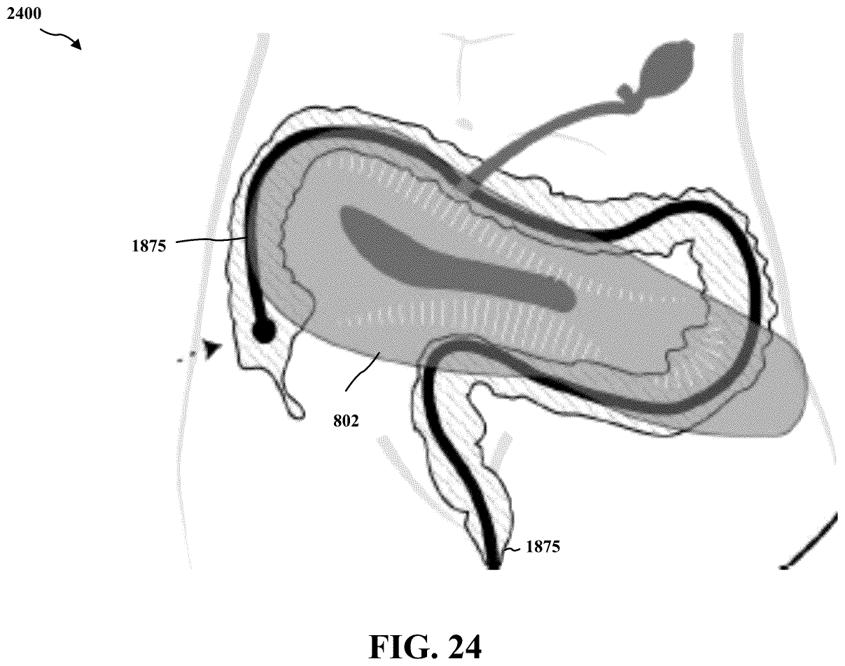

FIGS. 18-24 illustrate insertion of an endoscope and the assistance provided by the insert. For example, FIG. 18 illustrates a view 1800, and an expanded view 1850, showing that the insert blocks the endoscope 1875 from pushing the sigmoid colon upwards and creating a loop, e.g., as shown at 1877. The white striping in FIGS. 18-25 shows raised, sculpted or contoured areas such as shown in the various views presented herein. The inner core (e.g., 906) presses inward toward the patient as the bladder is inflated, and this can adjust the guidance of the endoscope 1875 that is provided by the insert. FIG. 18 also includes a side view 1825 of the insert 802 showing the curvature along the lower abdomen when positioned at the patient, e.g., such as positioned along under at least the secondary straps of the compression device. FIG. 19 illustrates a view 1900 showing a closer view of the endoscope 1875 being guided, or blocked, by the contouring of the insert 802 as the endoscope is moves through the sigmoid colon region of the patient. FIG. 20 illustrates a view 2000 showing the continued support and guidance provided as the endoscope 1875 is moved along, e.g., inserted along, the colon, following the view shown in FIGS. 18 and 19. FIG. 21 illustrates a view 2100 showing the continued support and guidance provided as the endoscope 1875 is moved along, e.g., inserted along, the colon, following the view shown in FIG. 20. For example, FIG. 21 illustrates that as the endoscope 1875 moves through the transverse colon, the insert 802 blocks (e.g., with the contour shown at 2177) the endoscope from pushing the colon down and forming an upper loop. FIG. 22 illustrates a view 2200 showing the continued support and guidance at 2277. FIG. 23 illustrates a view 2300 showing that the curved end helps to continue to guide movement of the endoscope 1875 as the endoscope moves past the position shown in FIG. 22. FIG. 24 illustrates a view 2400 showing that the endoscope 1875 reaches the cecum with reduced colon distortion due to the insert 802.

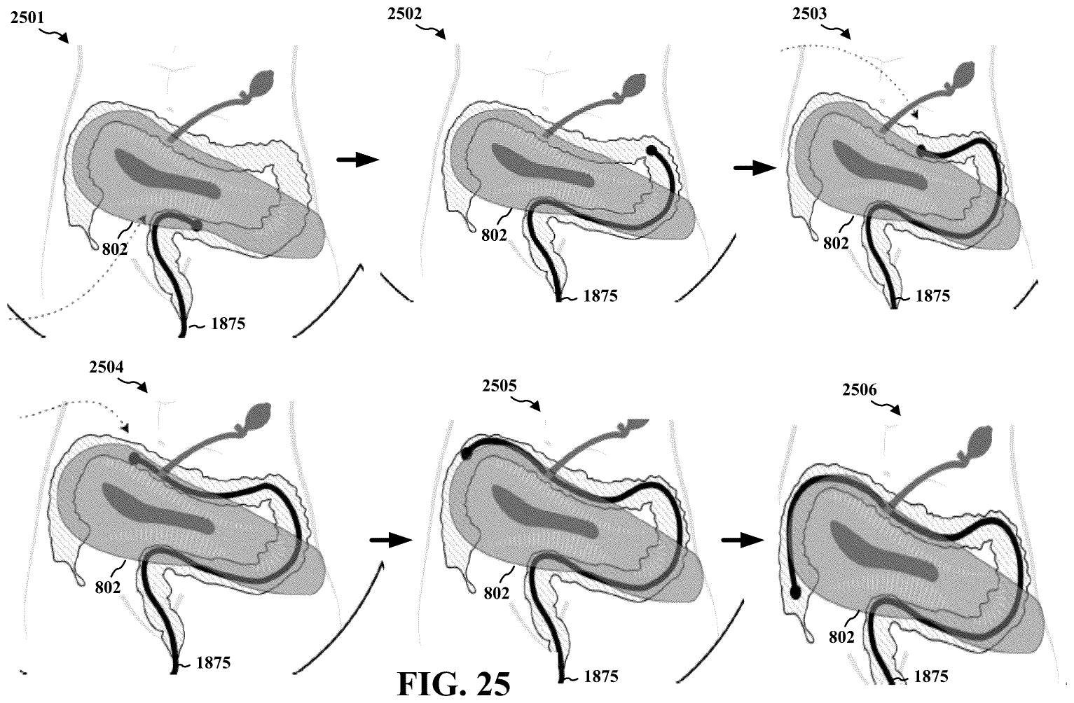

FIG. 25 illustrates views at different times during endoscope insertion and the assistance and guidance provided by the insert 802. For example, the views 2501, 2502, 2503, 2504, 2505, and 2506 show a progression in time during movement of the scope.

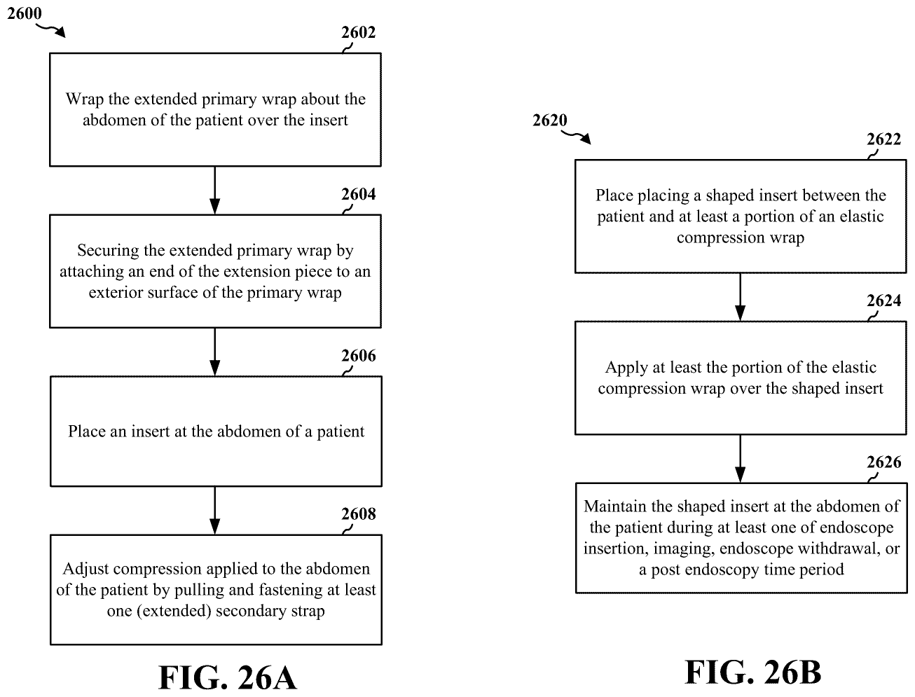

FIG. 26A illustrates a flowchart 2600 of a method of applying an insert with an endoscopy support device. At 2602, a user wraps a primary wrap about the abdomen of the patient, e.g., as described in connection with any of FIGS. 2-7. The user secures the primary wrap at 2604, by attaching an end of the extension piece to an exterior surface of the primary wrap. At 2606, the user places an insert at the abdomen of a patient. The insert may be placed, e.g., as described in connection with any of FIGS. 8A-25. The insert may be a shaped or contoured insert having an inner core with a harder material and an outer surface with a softer material. The shaped insert may be shaped to help prevent looping and to help guide an endoscope during movement within a colon. The insert may include any of the aspects described in connection with FIGS. 8A-25, for example. At 2608, the user adjusts the compression applied to the abdomen of the patient by pulling and fastening at least one secondary strap, e.g., as illustrated in FIG. 16 or 17. The user may readjust the pressure applied to the abdomen by adjusting one or more secondary strap. In some aspects, the insert may be applied to an abdomen of the patient prior to application of the primary wrap at 2602.

FIG. 26B illustrates a flowchart 2620 of a method of applying an insert with an endoscopy support device. The method illustrated in FIG. 26B may further include any of the aspects described in connection with the flowchart in FIG. 26A, and the method illustrated in FIG. 26A may further include any of the aspects described in connection with FIG. 26B, for example. The method may provide added, external support and guidance for movement of an endoscope during an endoscopy procedure. The method is a method of applying an insert at a patient in connection with an endoscopy procedure, and includes placing a shaped insert between the patient and at least a portion of an elastic compression wrap, as shown at 2622 in FIG. 26B. In some aspects, the elastic compression wrap is an example of an endoscopy support device and may be interchangeably referred to as an endoscopy support device. The insert may include a main body having a curved surface configured to extend across and curve along at least a portion of an abdomen of the patient; and at least one contour in an exterior of the main body. In some aspects, 2622 may correspond to 2606 in FIG. 26A.

As shown at 2624, the method may include applying at least the portion of the elastic compression wrap over the shaped insert. For example. FIG. 16 illustrates an example of the secondary straps being wrapped over the shaped insert. In some aspects, 2624 may correspond to 2608 in FIG. 26A.

As shown at 2626, the method may include maintaining the shaped insert at the abdomen of the patient during at least one of endoscope insertion, imaging, endoscope withdrawal, or a post endoscopy time period.

In some aspects, the method may include wrapping a primary wrap of the elastic compression wrap around the abdomen of the patient before placing the insert on an exterior side of the primary wrap, wherein the insert is placed between the primary wrap and at least one secondary strap of the elastic compression wrap, e.g., such as illustrated in FIGS. 14-15.

In some aspects, the method may include adjusting compression applied to the abdomen of the patient by fastening the at least one secondary strap of the elastic compression wrap over the shaped insert, e.g., such as illustrated in FIGS. 16-17.

In some aspects, the method may include placing a pressure sensor between the abdomen of the patient and the elastic compression wrap. FIGS. 13 and 14 illustrate examples aspects of applying a pressure sensing mat, as one example of a sensor.

In some aspects, the at least one contour includes: at least one shaped contouring along at least one side of the main body and configured to extend along at least one of a sigmoid colon region or a transverse colon region when placed at the patient and to guide movement of an endoscope along the transverse colon region or the sigmoid colon region. FIGS. 8A-12 and 14, 15, and 17-25 illustrate example aspects of a contoured insert, or endoscopy support device.

In some aspects, the at least one contour includes: a first concave contouring at a first side of the main body, wherein the first concave contouring is configured to extend along a sigmoid colon region when placed at the abdomen of the patient and is shaped to guide movement of an endoscope through the sigmoid colon region. FIGS. 18, 19, 20, and 25 illustrate example aspects of such a contour. Additionally, or alternatively, in some aspects, the at least one contour includes a second concave contouring at a second side of the main body, wherein the second concave contouring is configured to extend along a transverse colon region when placed at the abdomen of the patient and is shaped to guide the movement of the endoscope through the transverse colon region. FIGS. 21-25 illustrate example aspects of such a contour.

In some aspects, the at least one contour of the main body includes a tapered shape having a first end for a first placement toward a right hip of the patient and a second end for a second placement toward a left hip of the patient, wherein the first end is larger than the second end, and the main body tapers toward the second end. FIGS. 15 and 18-25 illustrate examples of the tapering shape.

In some aspects, the main body comprises a silicon material and a shaped inner core having a material with an increased hardness in comparison to the silicon material of the main body. In some aspects, the shaped inner core includes: a first contouring along a first side, wherein the first contouring is shaped to guide movement of an endoscope through a sigmoid colon region; and a second contouring along a second side, wherein the second contouring is shaped to guide the movement of the endoscope through a transverse colon region. FIGS. 8A-11, 14, 15, and 18-25 illustrate examples aspects of a shaped inner core.

In some aspects, the method may further include inflating an inflatable bladder positioned within the main body to adjust targeted pressure to the abdomen of the patient, wherein the inflatable bladder comprises a different material than the silicon material. FIG. 8B illustrates an example of inflating a bladder within an example insert.

The insert may be used, or applied, in any of various potential ways. In some aspects, the use of the insert may be particular to a particular patient and/or a particular part of the procedure. For example the insert may be applied pre-procedurally, intra-procedurally, during either an endoscope insertion phase, an imaging phase, and/or an endoscope withdrawal phase. The insert may be applied, or adjusted, at any point of the endoscopy procedure. Adjustments to compression may be delivered via the insert through adjustment of the compression device (e.g., the primary band and/or secondary straps) and/or adjustment of any insert component or feature designed to offer adjustability (e.g. placement and/or inflation or deflation of an air bladder). In some aspects, the method may include iterative application and full removal of the insert at various times during the endoscopy procedure (e.g, application, removal, application, removal, application, removal, and so forth). As an example, the insert may be applied while the endoscope is being advance, and then removed. The insert may be reapplied when the endoscope is advanced again, and/or when the endoscope is withdrawn. As another example, the insert may be applied and removed to obtain various images or views of the colon.

In some aspects, maintaining the compression applied by the device during the withdrawal phase of the procedure and while imaging is performed may help improve the detection of adenoma. The device may be maintained on the patient to reduce the common post-procedure complications of bloating and abdominal pain caused by bloating. Otherwise known as gaseous distention, bloating occurs following endoscopy procedures because physicians often use compressed air or carbon dioxide to insufflate parts of the bowel that are difficult to see and examine. The gas opens up the area to allow for a more complete visualization, enhancing the efficacy of the procedure. However, the gas also remains in the patient until it is either absorbed or expelled. Expulsion is the primary gas removal mechanism as absorption is a very inefficient process. Gaseous distention is a primary post-procedure complication and a frequent complaint from patients. However, when the wrap described herein remains in place after the procedure, the lower abdominal compression generated by the device allows the bowel to more rapidly evacuate trapped by directing excess gas towards the rectum. As a result, the severity and duration of post-procedure bloating and associated abdominal pain may be reduced.

In some aspects, an apparatus for placement at an abdomen of a patient in connection with an endoscopy procedure, includes a main body having a curved surface, wherein the main body is configured to extend across and curve along at least a portion of the abdomen of the patient; and at least one contour in an exterior of the main body, wherein the at least one contour is shaped to guide movement of an endoscope during one or more of insertion or withdrawal of the endoscopy procedure. FIGS. 8A-12 and 14, 15, and 17-25 illustrate example aspects of a contoured insert, or endoscopy support device.

In some aspects, the at least one contour includes: at least one shaped contouring along at least one side of the main body and configured to extend along at least one of a sigmoid colon region or a transverse colon region when placed at the patient and to guide the movement of the endoscope along the transverse colon region or the sigmoid colon region. In some aspects, the at least one contour includes: a first concave contouring at a first side of the main body, wherein the first concave contouring is configured to extend along a sigmoid colon region when placed at the abdomen of the patient and is shaped to guide movement of an endoscope through the sigmoid colon region. FIGS. 18, 19, 20, and 25 illustrate example aspects of such a contour. Additionally, or alternatively, in some aspects, the at least one contour includes a second concave contouring at a second side of the main body, wherein the second concave contouring is configured to extend along a transverse colon region when placed at the abdomen of the patient and is shaped to guide the movement of the endoscope through the transverse colon region. FIGS. 21-25 illustrate example aspects of such a contour.

In some aspects, the at least one contour of the main body includes a tapered shape having a first end (e.g., a first rounded end) for a first placement toward a right hip of the patient and a second end (e.g., a second rounded end) for a second placement toward a left hip of the patient, wherein the first end is larger than the second end, and the main body tapers toward the second end. FIGS. 15 and 18-25 illustrate examples of the tapering shape. In some aspects, the curved surface of the main body has a curved shape at a side to be placed toward the abdomen of the patient, and wherein the main body further comprises a convex portion at a second side of the main body to be placed away from the abdomen of the patient.

In some aspects, the main body comprises a silicon material and a shaped inner core having a material with an increased hardness in comparison to the silicon material of the main body. In some aspects, the shaped inner core includes: a first contouring along a first side, wherein the first contouring is shaped to guide movement of an endoscope through a sigmoid colon region; and a second contouring along a second side, wherein the second contouring is shaped to guide the movement of the endoscope through a transverse colon region. FIG. 8A-11, 14, 15, and 18-25 illustrate examples aspects of a shaped inner core.

In some aspects, the apparatus includes an inflatable bladder positioned within the main body, wherein the inflatable bladder comprises a different material than the silicon material, and wherein the inflatable bladder is positioned within the main body to provide a targeted increase of pressure to the abdomen of the patient when the apparatus is between the abdomen and an elastic compression wrap.

In some aspects, the apparatus includes at least one strap extending from the apparatus having a length to extend around the abdomen of the patient to hold the placement of the apparatus at the patient. FIG. 12 illustrates an example of such a strap/straps.

Example aspects of the present invention have now been described in accordance with the above advantages. It will be appreciated that these examples are merely illustrative of aspects of the present invention. Many variations and modifications will be apparent to those skilled in the art.

It is understood that the specific order or hierarchy of steps in the processes disclosed is an illustration of exemplary approaches. Based upon design preferences, it is understood that the specific order or hierarchy of steps in the processes may be rearranged. Further, some steps may be combined or omitted. The accompanying method claims present elements of the various steps in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects.” Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

The following aspects are illustrative only and may be combined with other aspects or teachings described herein, without limitation.

Aspects 1 is a shaped insert, or endoscopic support device, for placement at an abdomen of a patient in connection with an endoscopy procedure, comprising: a main body having a curved surface, wherein the main body is configured to extend across and curve along at least a portion of the abdomen of the patient; and at least one contour in an exterior of the main body, wherein the at least one contour is shaped to guide movement of an endoscope during one or more of insertion or withdrawal of the endoscopy procedure.

Aspect 2 is apparatus for placement at an abdomen of a patient in connection with an endoscopy procedure, comprising: a main body having a curved surface, wherein the main body is configured to extend across and curve along at least a portion of the abdomen of the patient; and at least one contour in an exterior of the main body, wherein the at least one contour is shaped to guide movement of an endoscope during one or more of insertion or withdrawal of the endoscopy procedure.

In aspect 3, the apparatus of aspect 1 or aspect 2 further includes that the at least one contour includes: at least one shaped contouring along at least one side of the main body and configured to extend along at least one of a sigmoid colon region or a transverse colon region when placed at the patient and to guide the movement of the endoscope along the transverse colon region or the sigmoid colon region.

In aspect 4, the apparatus of any of aspects 1-3 further includes that the at least one contour includes: a first concave contouring at a first side of the main body, wherein the first concave contouring is configured to extend along a sigmoid colon region when placed at the abdomen of the patient and is shaped to guide the movement of the endoscope through the sigmoid colon region; and a second concave contouring at a second side of the main body, wherein the second concave contouring is configured to extend along a transverse colon region when placed at the abdomen of the patient and is shaped to guide the movement of the endoscope through the transverse colon region.

In aspect 5, the apparatus of any of aspects 1-4 further includes that the at least one contour of the main body includes a tapered shape having a first end for a first placement toward a right hip of the patient and a second end for a second placement toward a left hip of the patient, wherein the first end is larger than the second end, and the main body tapers toward the second end.

In aspect 6, the apparatus of aspect 5 further includes that the curved surface of the main body has a curved shape at a side to be placed toward the abdomen of the patient, and wherein the main body further comprises a convex portion at a second side of the main body to be placed away from the abdomen of the patient.

In aspect 7, the apparatus of any of aspects 1-6 further includes that the main body comprises a silicon material.

In aspect 8, the apparatus of any of aspects 1-7 further includes that the apparatus further includes: an inflatable bladder positioned within the main body, wherein the inflatable bladder comprises a different material than the silicon material, and wherein the inflatable bladder is positioned within the main body to provide a targeted increase of pressure to the abdomen of the patient when the apparatus is between the abdomen and an elastic compression wrap.

In aspect 9, the apparatus of aspect 7 or 8 further includes that the apparatus further includes a shaped inner core having a material with an increased hardness in comparison to the silicon material of the main body.

In aspect 10, the apparatus of aspect 9 further includes that the shaped inner core includes: a first contouring along a first side, wherein the first contouring is shaped to guide the movement of the endoscope through a sigmoid colon region; and a second contouring along a second side, wherein the second contouring is shaped to guide the movement of the endoscope through a transverse colon region.

In aspect 11, the apparatus of any of aspects 1-10 further includes at least one strap extending from the apparatus having a length to extend around the abdomen of the patient to hold the placement of the apparatus at the patient.

Aspect 12 is a method of applying an insert, or endoscopic support apparatus, at a patient in connection with an endoscopy procedure, the method comprising: placing a shaped insert between the patient and at least a portion of an elastic compression wrap, wherein the shaped insert includes: a main body having a curved surface configured to extend across and curve along at least a portion of an abdomen of the patient; and at least one contour in an exterior of the main body; applying at least the portion of the elastic compression wrap over the shaped insert; and maintaining the shaped insert at the abdomen of the patient during at least one of endoscope insertion, imaging, endoscope withdrawal, or a post endoscopy time period.

In aspect 13, the method of aspect 12 further includes wrapping a primary wrap of the elastic compression wrap around the abdomen of the patient before placing the insert on an exterior side of the primary wrap, wherein the insert is placed between the primary wrap and at least one secondary strap of the clastic compression wrap.

In aspect 14, the method of aspect 12 or 13 further includes adjusting compression applied to the abdomen of the patient by fastening the at least one secondary strap of the elastic compression wrap over the shaped insert.

In aspect 15, the method of any of aspects 12-14 further includes placing a pressure sensor between the abdomen of the patient and the elastic compression wrap.

In aspect 16, the method of any of aspects 12-15 further includes that the at least one contour includes: at least one shaped contouring along at least one side of the main body and configured to extend along at least one of a sigmoid colon region or a transverse colon region when placed at the patient and to guide movement of an endoscope along the transverse colon region or the sigmoid colon region.

In aspect 17, the method of any of aspects 12-16 further includes that the at least one contour includes: a first concave contouring at a first side of the main body, wherein the first concave contouring is configured to extend along a sigmoid colon region when placed at the abdomen of the patient and is shaped to guide movement of an endoscope through the sigmoid colon region; and a second concave contouring at a second side of the main body, wherein the second concave contouring is configured to extend along a transverse colon region when placed at the abdomen of the patient and is shaped to guide the movement of the endoscope through the transverse colon region.

In aspect 18, the method of any of aspects 12-17 further includes that the at least one contour of the main body includes a tapered shape having a first end for a first placement toward a right hip of the patient and a second end for a second placement toward a left hip of the patient, wherein the first end is larger than the second end, and the main body tapers toward the second end.

In aspect 19, the method of any of aspects 12-18 further includes that the main body comprises a silicon material and a shaped inner core having a material with an increased hardness in comparison to the silicon material of the main body.

In aspect 20, the method of any of aspects 12-19 further includes that the shaped inner core includes: a first contouring along a first side, wherein the first contouring is shaped to guide movement of an endoscope through a sigmoid colon region; and a second contouring along a second side, wherein the second contouring is shaped to guide the movement of the endoscope through a transverse colon region.

In aspect 21, the method of any of aspects 12-20 further includes inflating an inflatable bladder positioned within the main body to adjust targeted pressure to the abdomen of the patient, wherein the inflatable bladder comprises a different material than the silicon material.

1. An apparatus for placement at an abdomen of a patient in connection with an endoscopy procedure, comprising:
a main body having a curved surface, wherein the main body is configured to extend across and curve along at least a portion of the abdomen of the patient; and
at least one contour in an exterior of the main body, wherein the at least one contour is shaped to guide movement of an endoscope during one or more of insertion or withdrawal of the endoscopy procedure.

2. The apparatus of claim 1, wherein the at least one contour includes:
at least one shaped contouring along at least one side of the main body and configured to extend along at least one of a sigmoid colon region or a transverse colon region when placed at the patient and to guide the movement of the endoscope along the transverse colon region or the sigmoid colon region.

3. The apparatus of claim 1, wherein the at least one contour includes:
a first concave contouring at a first side of the main body, wherein the first concave contouring is configured to extend along a sigmoid colon region when placed at the abdomen of the patient and is shaped to guide the movement of the endoscope through the sigmoid colon region; and
a second concave contouring at a second side of the main body, wherein the second concave contouring is configured to extend along a transverse colon region when placed at the abdomen of the patient and is shaped to guide the movement of the endoscope through the transverse colon region.

4. The apparatus of claim 1, wherein the at least one contour of the main body includes a tapered shape having a first end for a first placement toward a right hip of the patient and a second end for a second placement toward a left hip of the patient, wherein the first end is larger than the second end, and the main body tapers toward the second end.

5. The apparatus of claim 4, wherein the curved surface of the main body has a curved shape at a side to be placed toward the abdomen of the patient, and wherein the main body further comprises a convex portion at a second side of the main body to be placed away from the abdomen of the patient.

6. The apparatus of claim 1, wherein the main body comprises a silicon material.

7. The apparatus of claim 6, wherein the apparatus further includes:
an inflatable bladder positioned within the main body, wherein the inflatable bladder comprises a different material than the silicon material, and wherein the inflatable bladder is positioned within the main body to provide a targeted increase of pressure to the abdomen of the patient when the apparatus is between the abdomen and an elastic compression wrap.

8. The apparatus of claim 6, wherein the apparatus further includes a shaped inner core having a material with an increased hardness in comparison to the silicon material of the main body.

9. The apparatus of claim 8, wherein the shaped inner core includes:
a first contouring along a first side, wherein the first contouring is shaped to guide the movement of the endoscope through a sigmoid colon region; and
a second contouring along a second side, wherein the second contouring is shaped to guide the movement of the endoscope through a transverse colon region.

10. The apparatus of claim 1, further comprising at least one strap extending from the apparatus having a length to extend around the abdomen of the patient to hold the placement of the apparatus at the patient.

11. A method of applying an insert at a patient in connection with an endoscopy procedure, the method comprising:
placing a shaped insert between the patient and at least a portion of an elastic compression wrap, wherein the shaped insert includes:
a main body having a curved surface configured to extend across and curve along at least a portion of an abdomen of the patient; and
at least one contour in an exterior of the main body;
applying at least the portion of the elastic compression wrap over the shaped insert; and
maintaining the shaped insert at the abdomen of the patient during at least one of endoscope insertion, imaging, endoscope withdrawal, or a post endoscopy time period.

12. The method of claim 11, further comprising:
wrapping a primary wrap of the elastic compression wrap around the abdomen of the patient before placing the insert on an exterior side of the primary wrap, wherein the insert is placed between the primary wrap and at least one secondary strap of the elastic compression wrap.

13. The method of claim 12, further comprising:
adjusting compression applied to the abdomen of the patient by fastening the at least one secondary strap of the elastic compression wrap over the shaped insert.

14. The method of claim 11, further comprising:
placing a pressure sensor between the abdomen of the patient and the elastic compression wrap.

15. The method of claim 11, wherein the at least one contour includes:
at least one shaped contouring along at least one side of the main body and configured to extend along at least one of a sigmoid colon region or a transverse colon region when placed at the patient and to guide movement of an endoscope along the transverse colon region or the sigmoid colon region.

16. The method of claim 11, wherein the at least one contour includes:
a first concave contouring at a first side of the main body, wherein the first concave contouring is configured to extend along a sigmoid colon region when placed at the abdomen of the patient and is shaped to guide movement of an endoscope through the sigmoid colon region; and
a second concave contouring at a second side of the main body, wherein the second concave contouring is configured to extend along a transverse colon region when placed at the abdomen of the patient and is shaped to guide the movement of the endoscope through the transverse colon region.

17. The method of claim 11, wherein the at least one contour of the main body includes a tapered shape having a first end for a first placement toward a right hip of the patient and a second end for a second placement toward a left hip of the patient, wherein the first end is larger than the second end, and the main body tapers toward the second end.

18. The method of claim 11, wherein the main body comprises a silicon material and a shaped inner core having a material with an increased hardness in comparison to the silicon material of the main body.

19. The method of claim 18, wherein the shaped inner core includes:
a first contouring along a first side, wherein the first contouring is shaped to guide movement of an endoscope through a sigmoid colon region; and
a second contouring along a second side, wherein the second contouring is shaped to guide the movement of the endoscope through a transverse colon region.

20. The method of claim 18, wherein the method further includes:
inflating an inflatable bladder positioned within the main body to adjust targeted pressure to the abdomen of the patient, wherein the inflatable bladder comprises a different material than the silicon material.
